(19) United States
(12) Patent Application Publication
(10) Pub. No.: US 2025/0072939 A1
(43) Pub. Date: Mar. 6, 2025

(54) Percutaneous Minimally Invasive Cross Connector System

(71) Applicants: Oluwatodimu Richard Raji, San Francisco, CA (US); Dimitriy Kondrashov, San Francisco, CA (US)

(72) Inventors: Oluwatodimu Richard Raji, San Francisco, CA (US); Dimitriy Kondrashov, San Francisco, CA (US)

(21) Appl. No.: 18/458,000

(22) Filed: Aug. 29, 2023

(51) Int. Cl. A61B 17/70

(52) U.S. Cl. CPC A61B 17/708; A61B 17/7082; A61B 17/7083; A61B 2017/00477

(57) ABSTRACT

A cross connector device is provided. The cross connector device comprises a portal having a rod attachment feature with a circular cutout, a tulip attached to the portal, a cannula detachably attached to the tulip, and a retention component detachably connected to the portal.

### FIELD OF THE INVENTION

The present invention relates generally to a medical device. More specifically, the present invention is a minimally invasive cross connector.

### BACKGROUND OF THE INVENTION

The minimally invasive or percutaneous placement of pedicle screws is becoming a larger part of spinal surgery. The indications include backing up a long construct after multilevel XLIF or ALIF surgery, the minimally invasive treatment of spine fractures with or without neurological deficits, and the stabilization of an unstable spine due to metastatic disease. It is becoming very popular in repairing unstable 3 column chance fractures and ankylosed spine for patients with ankylosing spondylitis, diffuse idiopathic skeletal hyperostosis, or ankylosis due to advanced degenerative disc disease. Minimally invasive spinal surgery results in less physiological trauma to the patient during the operation, faster recovery, and a smaller chance of infection based on published peer-reviewed studies across the past several years.

The limitation of percutaneous fixation is the inability to place a cross-link to connect the rods together. The use of a cross-link is widespread in open thoracolumbar fusion and instrumentation. A cross-link is used to increase the rigidity of the construct, especially in osteoporotic bone. It is especially useful for long constructs and bone compromised by osteopenia or osteoporosis. Placing a cross-link creates a fixed angle construct and decreases the chance of screw pullout, especially at the cephalad and caudal ends of the long construct. The inability to place a cross-link in a percutaneous construct is a significant limitation. Such an inability in those situations can lead to construct failure due to screw pullout and the weakened rigidity of the construct in highly unstable spine fractures. Placing a cross-link in those situations requires opening the incision and placing a cross-link, which defeats the purpose of minimally invasive or percutaneous screw fixation.

The present invention is intended to address problems associated with and/or otherwise improve on conventional devices through an innovative device designed to provide a convenient, effective means for minimally invasive spine surgery while incorporating other problem-solving features.

### SUMMARY OF THE INVENTION

This summary is provided to introduce a selection of concepts in a simplified form, that are further described below in the Detailed Description. This summary is not intended to identify key features or essential features of the claimed subject matter. Nor is this summary intended to be used to limit the claimed subject matter's scope.

According to some embodiments, a cross connector device is disclosed. The cross connector device comprises a portal having a rod attachment feature with a circular cutout, a tulip attached to the portal, a cannula detachably attached to the tulip, and a retention component detachably connected to the portal.

In some embodiments, the portal of the present invention may include a plurality of lips and the cannula that includes two tabs.

In some embodiments, the retention component of the present invention may include helical internal threads.

In some other embodiments, the retention component may also include a plurality of lips.

### BRIEF DESCRIPTION OF THE DRAWINGS

The accompanying drawings, which are incorporated in and constitute a part of this disclosure, illustrate various embodiments of the present disclosure.

Furthermore, the drawings may contain text or captions that may explain certain embodiments of the present disclosure. This text is included for illustrative, non-limiting, explanatory purposes of certain embodiments detailed in the present disclosure.

FIG. 1 is an illustration of one embodiment of the present invention.

FIG. 2 is an illustration of one embodiment of the present invention without the retention component.

FIG. 3 is an illustration of one embodiment of the present invention connected with rods.

FIG. 4 is an illustration of one embodiment of the present invention with the retention component installed.

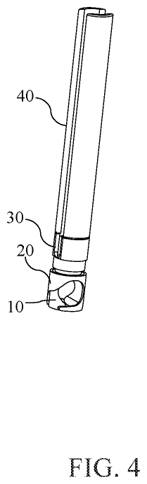

FIG. 5 is a front view of one embodiment of the portal and the tulip of the present invention.

FIG. 6 is a rear view of one embodiment of the portal and the tulip of the present invention.

FIG. 7 is a left side view of one embodiment of the portal and the tulip of the present invention.

FIG. 8 is a bottom view of one embodiment of the portal and the tulip of the present invention.

FIG. 9 is an illustration of one embodiment of the retention component of the present invention.

FIG. 10 is a front view of one embodiment of the portal, the tulip and the cannula of the present invention.

FIG. 11 is a rear view of one embodiment of the portal, the tulip and the cannula of the present invention.

FIG. 12 is a left side view of one embodiment of the portal, the tulip and the cannula of the present invention.

FIG. 13 is a right side view of one embodiment of the portal, the tulip and the cannula of the present invention.

### DETAIL DESCRIPTIONS OF THE INVENTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the appended claims. The present disclosure contains headers. It should be understood that these headers are used as references and are not to be construed as limiting upon the subjected matter disclosed under the header.

The present invention describes a system and method for both open and percutaneous top-loading cross-linking as an adjunct to existing open and percutaneous spinal rod fixation constructs.

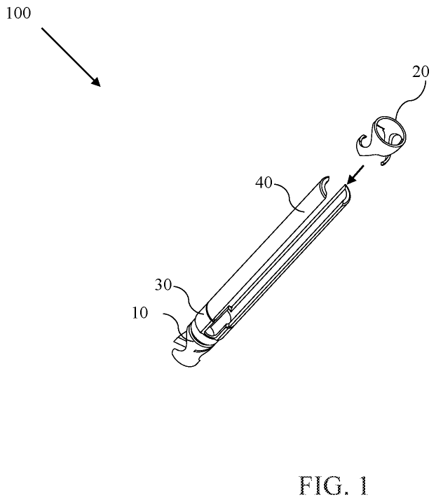

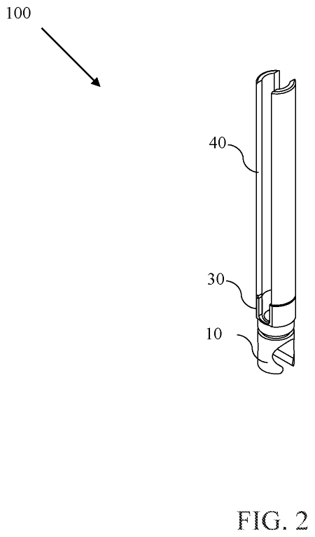

As shown in FIG. 1 and FIG. 2, the present invention provides a cross connector device 100 comprising a portal 10, a retention component 20, a tulip 30, and a cannula 40. The portal 10 can be attached to the tulip 30, which can be attached to the cannula 40. The retention component 20 can be attached to the portal 10 to form the present invention.

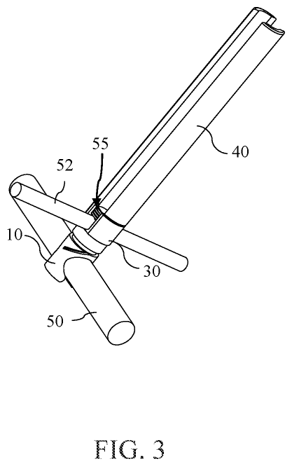

As shown in FIG. 3, the portal 10 can be affixed to the tulip 30 with the cannula (extended tabs) 40 and onto an existing rod construct 50, after which a rod (cross-connecting rod) 52, bar, rail, or plate can be installed onto the tulip to cross-connect multiple rods.

The cross-connecting rod 52 can then be kept in place using a retention cap 55. This system may or may not require any additional incisions apart from those already created.

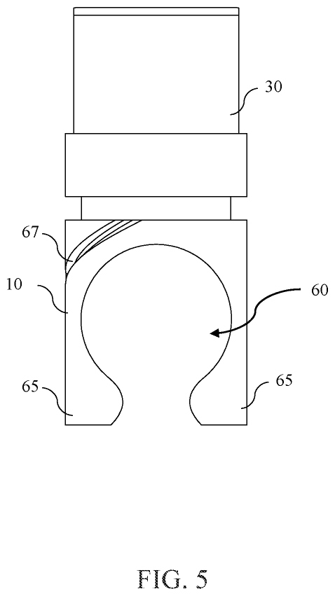

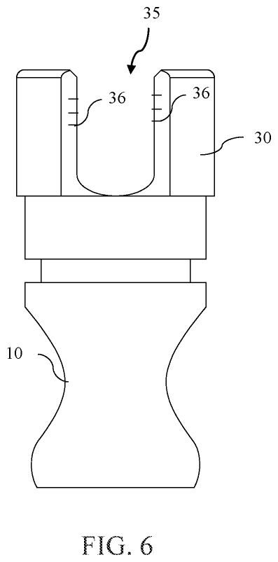

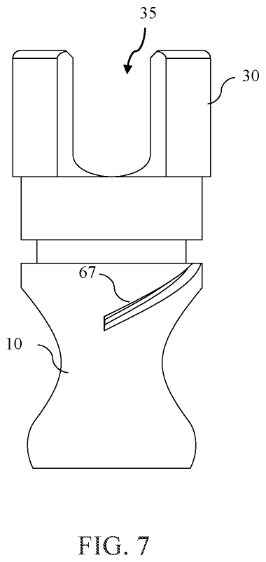

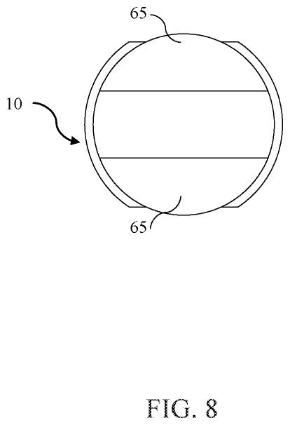

FIGS. 5-8 display embodiments and views of the portal 10. The portal 10 can comprise a rod attachment feature with a circular cutout 60 to hook or clip onto a primary fixation rod (an existing rod construct) 50. The cutout 60 may be circular, elliptical, rectangular, polygonal, triangular, or a combination of shapes. The cutout 60 may also implement multiple sized shapes to make it adaptable to various rod sizes.

In one embodiment, this attachment feature may be implemented by hooking or clipping it onto the fixation rod 50 upon the application of radial or axial forces toward the fixation rod 50.

Lip(s) 65 may be employed for rod retention. If attached to a fixation rod 50 with a circular cross-section, the fixation rod 50 may be used in conjunction with the portal 10 to provide an axis of rotation for direct customizable orientation of the portal 10 and indirect orientation of the cross-connecting rod 52, rail, bar, or plate. This feature may also implement helical and/or revolved male/female external threads 67 which may or may not be tapered and may be interfaced with a retention component 20 to radially clamp onto the fixation rod 50 upon the application of axial and/or rotational loads.

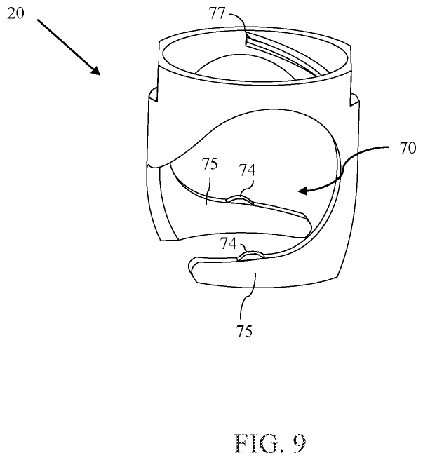

FIG. 9 shows the retention component 20. The retention component 20 can be detachable and attaches to the portal 10. In the embodiment described, the retention component 20 relies on helical and/or revolved circular cutouts 70, acting as a female mating construct to accommodate the fixation rod 50 while providing a direct or indirect (through the portal 10) radial and/or axial clamping force onto the fixation rod 50 for retention and additional stability upon the application of axial and/or torsional loads through its driving feature(s). Thus, the retention component can be rotated and pushed downwards from the top of the present invention to be attached to the portal through the thread engagement.

The circular cutout(s) 70 on this retention component 20 may also feature a lip(s) 75 with one or more bumps 74 on the lips (single or both lips for a snap attachment) 75 for additional retention onto the primary fixation rod 50. While a single circular cut out 70 (with rod drive cutout for inserting the fixation rod 50) is shown for a circular rod, multiple cut outs may be used to accommodate the varying shapes and sizes of fixation rods 50.

Additionally, the retention component 20 may also implement helical and/or revolved male/female internal threads 77, which may or may not be tapered and may be interfaced with the portal 10 upon the application of axial and/or rotational loads. At the same time, in some embodiments, the retention component 20 may also employ a threaded or non-threaded male mating construct similar to the threaded cap 55 or secondary rod/plate to provide a radial clamping force onto the primary fixation rod 50 and/or retention component 20 for additional retention upon the application of axial and/or torsional loads and through its driving feature(s).

FIG. 5 shows the tulip 30, which may be rigidly, semi rigidly, or flexibly combined with the portal 10. The tulip 30 is used as a seat for the cross-connecting rod 52, bar, rail, or plate. The tulip 30 can employ multiple cylindrical cutouts 35, following varying trajectories to provide allowance for variations in the angulation of the cross-connecting rod 52 or plate about the anterior-posterior axis upon implantation. While a circular/cylindrical cutout 35 profile is shown, the profile may be circular, elliptical, rectangular, polygonal, triangular, or a combination of these shapes. One or more of the cutouts 35 may also follow the same and/or different trajectories.

In some embodiments, The tulip 30 may include a threaded feature 36 above/within the cutout 35 to interface with a threaded cap 55 to provide a radial and/or axial clamping force onto the cross-connecting rod 52, rail, bar, or plate for retention upon the application of axial and/or torsional loads through its driving feature(s).

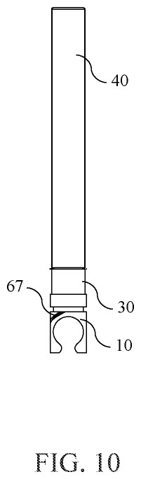

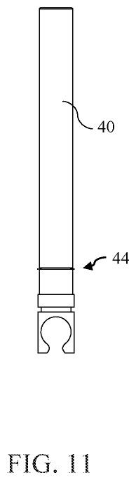

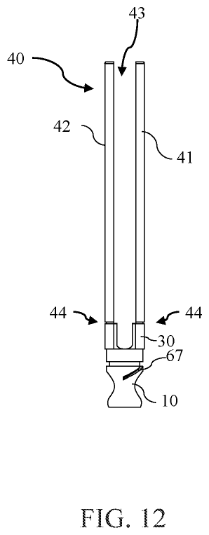

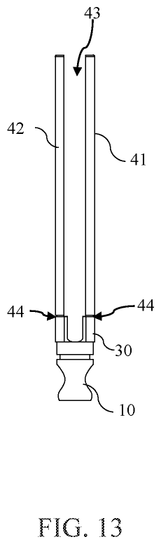

FIGS. 10-13, show the channel or cannula 40, which may comprise two or more detachable tabs 41, 42, extending from the tulip 30. The gap 43 between both tabs 41, 42 is designed to guide the installation of the cross-connecting rod 52. While the tabs 41, 42 shown are mostly independent of each other, both tabs 41, 42 may be connected to each other by means of a temporary threaded or unthreaded sleeve or a rigid/semirigid connection between both tabs. In one embodiment, notch features 44 are implemented at the cannula-tulip junction to facilitate the detachment of the cannula/working channel 40 from the tulip 30 upon application of bending or torsional loads.

While a single-bodied, straight cross-connecting rod 52 with a circular cross section can be used, the cross-connecting rod 52 can also be a rail, bar, or a plate; may be multi-bodied, curved, entailing circular, elliptical, rectangular, polygonal, or triangular; or may be a combination of the shapes in its cross-section.

Although the invention has been explained in relation to its preferred embodiment, it is to be understood that many other possible modifications and variations can be made without departing from the spirit and scope of the invention.

What is claimed is:

1. A cross connector device comprising:
a portal having a rod attachment feature with a circular cutout,
a tulip attached to the portal,
a cannula detachably attached to the tulip, and
a retention component detachably connected to the portal.

2. The cross connector device as claimed in claim 1, wherein the portal includes a plurality of lips.

3. The cross connector device as claimed in claim 1, wherein the cannula includes two tabs detachably connected to the tulip.

4. The cross connector device as claimed in claim 2, wherein the cannula includes a gap between the two tabs.

5. The cross connector device as claimed in claim 1, wherein the portal and the retention component includes helical internal threads.

6. The cross connector device as claimed in claim 2, wherein the retention component includes helical internal threads, which is tapered.

7. The cross connector device as claimed in claim 1, wherein the retention component includes a plurality of lips with one or more bumps placed on the lips.

8. The cross connector device as claimed in claim 2, wherein the retention component includes a plurality of lips.

9. A cross connector device comprising:
a portal having a rod attachment feature with a circular cutout, the portal includes two lips,
a tulip attached to the portal,
a cannula detachably attached to the tulip, and
a retention component detachably connected to the portal.

10. The cross connector device as claimed in claim 9, wherein the cannula includes two tabs detachably connected to the tulip.

11. The cross connector device as claimed in claim 10, wherein the cannula includes a gap between the two tabs.

12. The cross connector device as claimed in claim 9, wherein the portal and the retention component includes helical internal threads.

13. The cross connector device as claimed in claim 10, wherein the retention component includes helical internal threads, which is tapered.

14. The cross connector device as claimed in claim 9, wherein the retention component includes a plurality of lips with one or more bumps placed on the lips.

15. The cross connector device as claimed in claim 10, wherein the retention component includes a plurality of lips with one or more bumps placed on the lips.

16. A cross connector device comprising:
a portal having a rod attachment feature with a circular cutout, the portal includes two lips and helical internal threads,
a tulip attached to the portal,
a cannula detachably attached to the tulip, and
a retention component having a plurality of lips and helical internal threads, the retention component is detachably connected to the portal and includes helical internal threads.

17. The cross connector device as claimed in claim 16, wherein the portal includes a plurality of lips.

18. The cross connector device as claimed in claim 16, wherein the cannula includes two tabs detachably connected to the tulip.

19. The cross connector device as claimed in claim 17, wherein the cannula includes a gap between the two tabs.

20. The cross connector device as claimed in claim 17, wherein the retention component is tapered.
